(19) United States
(12) Patent Application Publication
(10) Pub. No.: US 2024/0122601 A1
(43) Pub. Date: Apr. 18, 2024

(54) EMBOLIC COIL DETACHMENT COUPLER MECHANISM

(71) Applicant: Kai Medtech LLC, Lake Forest, CA (US)

(72) Inventors: Nicholas Cavestany, Lake Forest, CA (US); George Martinez, Lake Forest, CA (US)

(73) Assignee: Kai Medtech LLC, Lake Forest, CA (US)

(21) Appl. No.: 18/380,192

(22) Filed: Oct. 15, 2023

Related U.S. Application Data
(60) Provisional application No. 63/416,517, filed on Oct. 15, 2022.

(51) Int. Cl. A61B 17/12

(52) U.S. Cl. CPC A61B 17/1214; A61B 2017/12054

(57) ABSTRACT

The present invention describes systems and methods using a detachment coupler mechanism of an implant assembly in delivering and detaching an implant at a target site or location within the neurovascular space/body of patient.

### CROSS-REFERENCE TO RELATED APPLICATIONS

This application claims the benefit of U.S. Provisional Application No. 63/416,517 filed Oct. 15, 2022, which is incorporated herein by reference.

### FIELD

The present invention relates to a biocompatible medical device for insertion into the body during medical procedures, and more specifically, delivering a medical device having a detachment coupler mechanism through a catheter in the human vasculatures to a target site or location within the neurovascular space for detaching an implant at the target site or location.

### BACKGROUND

Detachable implants can fail to detach or can detach prematurely/uncontrolled. Mechanical engagement in comparable devices require constant force applied by securing member and in certain configurations restrict rotation during advancement of the implant to the target site.

Accordingly, there is a need for a method of delivering and detaching an implant to a target site or location within the neurovascular space/body of patient. The present invention is directed toward systems and methods for a detachment mechanism that detaches through a mechanical release between the interlocking proximal end of the detachable implant coil and the distal end of a pusher wire.

### SUMMARY

The present invention pertains to systems and methods using a detachment coupler mechanism of an implant assembly in delivering and detaching an implant at a target site or location within the neurovascular space/body of patient. In the embodiments shown, the implant is an embolic coil. The detachment coupler mechanism described may be applied to the delivery of many other implants and is not limited to an embolic coil.

One embodiment of the present invention is a detachment coupler mechanism that includes a pusher coupled to an implant. The pusher is configured to advance the implant through a catheter and position the implant at the target site or location. The implant is then detached from detachment coupler mechanism at the target site or location.

### BRIEF DESCRIPTION OF THE DRAWINGS

FIG. 1 shows a perspective view of an embolic coil detachment coupler mechanism to detach an implant pusher from an implant assembly at a target location.

FIGS. 2A and 2B show an end view and side view of the distal end of the implant pusher having a detachment tip.

FIGS. 3A and 3B show an end view and side view of the proximal end of the implant assembly detachment coupler.

FIG. 4 is a sectional view showing the delivery configuration of the detachment coupler mechanism with the detachment tip coupled to the detachment coupler.

FIG. 5 is a sectional view showing the detachment configuration of the detachment coupler mechanism when the embolic coil is at the target site or location.

FIG. 6 is a sectional view showing the detachment tip detached from the implant.

FIGS. 7-9 are sectional views showing the connect/disconnect features of the detachment coupler mechanism that are configured to couple the detachment tip of the pusher with the detachment coupler of the implant.

### DETAILED DESCRIPTION

The present invention describes systems and methods using a detachment coupler mechanism of an implant assembly in delivering and detaching an implant at a target site or location within the neurovascular space/body of patient. In the embodiments shown, the implant is an embolic coil. The detachment coupler mechanism described may be applied to the delivery of many other implants and is not limited to an embolic coil.

In general, the detachment coupler mechanism includes the pusher and the implant. The pusher is configured to advance the implant through a catheter and position the implant at the target site or location. The implant is then detached from detachment coupler mechanism at the target site or location.

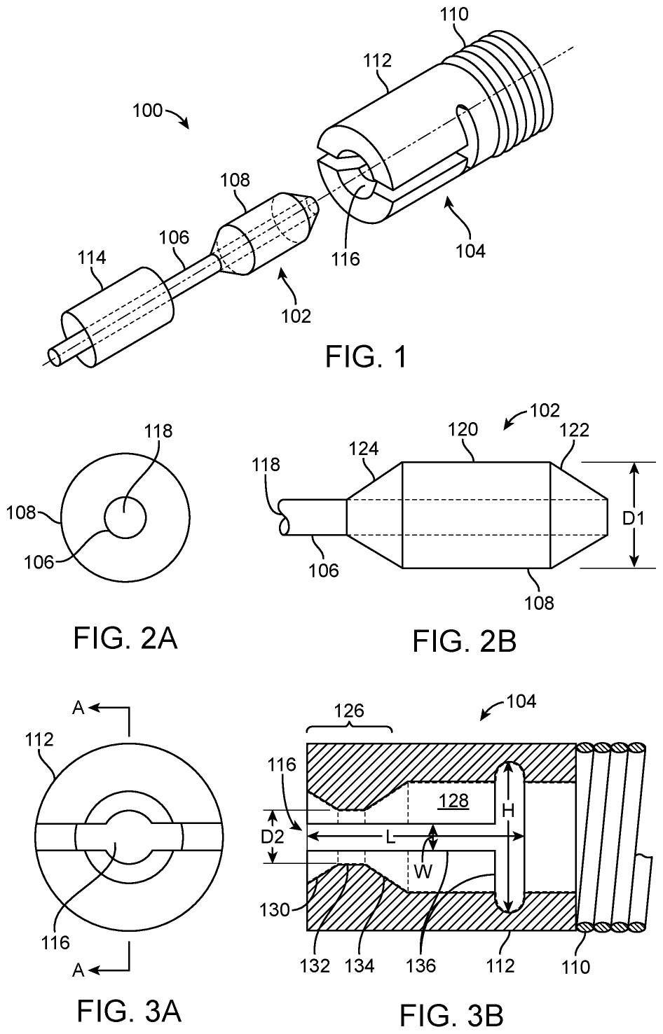

FIG. 1 shows an exploded perspective view of a detachment coupler mechanism 100 portion of an implant delivery device having a pusher 102 configured to be coupled with an implant 104. The pusher 102 includes a pusher wire 106 with a detachment tip 108 at a distal end. The implant 104 includes an embolic coil 110 with a detachment coupler 112 at a proximal end having a proximal opening 116 for insertion of the detachment tip 108. A puller 114 is configured to assist in detaching the detachment tip 108 from the detachment coupler 112, shown in FIG. 5.

When coupled, the implant 104 to pusher 102 interface of the detachment coupler mechanism 100 provides mechanical control of the engagement and disengagement of the implant 104 by retaining the detachment tip 108 within a body cavity of the detachment coupler 112. The detachment coupler mechanism 100 permits axial movement of the pusher 102 and implant 104 through a delivery catheter to the target site or location. The detachment method of the detachment coupler mechanism 100 uses a puller 114 that allows the user to mechanically control the disengagement of the pusher 102 and implant 104. Upon implant 110 detachment, the pusher 102 and puller 114 are withdrawn.

FIGS. 2A and 2B show an end view and a side view of a distal end portion of the pusher 102 including the pusher wire 106 with the detachment tip 108. The pusher wire 106 may be an elongated shaft having a distal end, a proximal end, and a lumen 118 through which a guidewire can pass. The pusher wire 106 has a length so that the proximal end extends from a proximal end of the delivery catheter for manipulation during delivery and detachment of the implant 104. The pusher wire 106 is made of a flexible material that can be advanced through a catheter and have sufficient axial strength to be able to push the implant 104 through the catheter.

The detachment tip 108 is designed for insertion and removal through the opening 116 of the detachment coupler 112. The detachment tip 108 is sized to fit within the delivery catheter and includes a detachment tip body 120 having a tapered distal end 122 and a tapered proximal end 124. The detachment tip body 120 may be various shapes. In the embodiment shown, the detachment tip body 120 is cylindrical in shape having a first diameter D1.

FIGS. 3A and 3B show an end view and a sectional side view A-A of a proximal end portion of the implant 104 including the embolic coil 110 coupled with the detachment coupler 112. The opening 116 on the proximal end of the detachment coupler 112 includes an inlet 126 leading to a detachment tip cavity 128. The inlet 126 includes a proximal ramped portion 130, a reduced diameter portion 132, and a distal ramped portion 134 coupled to the detachment tip cavity 128. The reduced diameter portion 132 has a second diameter of D2, which is less than first diameter D1 of the detachment tip body 120. The opening 116 configured to expand to an insertion position during insertion of the detachment tip 108 and retract once the detachment tip 108 is positioned in the detachment tip cavity 128.

The detachment coupler 112 may include one or more slots or cuts 136 to add a balance of retention strength and ease of detachment of the detachment tip 108. In the embodiments shown, the cuts 136 are “T” cuts on each side of the detachment coupler 112 having a length L, width W and height H. The force/friction strength of detachment can be modified by the length L of the “T” cut, with an increase with a shorter “T” cut 136 and a decrease with a longer “T” cut 136. The “T” cut 136 allows for the proximal opening 116 of the detachment coupler 112 to flex during retraction of the detachment tip 108, allowing for reliable detachment between the pusher 102 and implant 104.

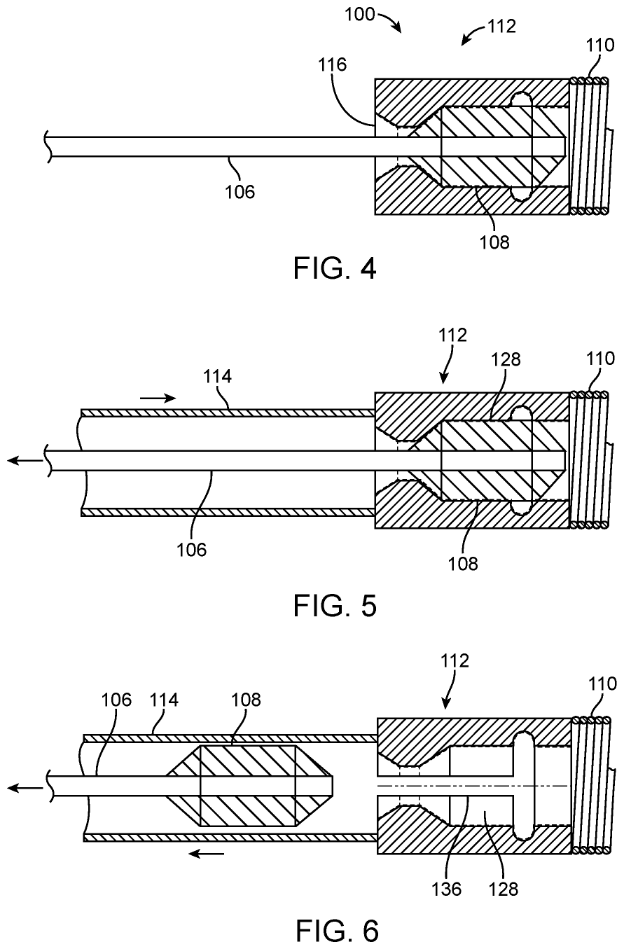

FIG. 4 is a sectional view showing the delivery configuration of the detachment coupler mechanism 100 with the detachment tip 108 coupled to the detachment coupler 112. The implant 104 to pusher 102 interface provides mechanical control of the engagement of the detachable implant or embolic coil 110 by retaining the detachment tip 108 within the detachment tip cavity 128 of the detachment coupler 112. The detachment tip 108 rotates freely within the detachment coupler 112 and does not require an external force to remain secured. The pusher wire 106 is moved or pushed distally to advance the embolic coil 110 to the target site or location.

FIG. 5 is a sectional view showing the detachment configuration of the detachment coupler mechanism 100 when the embolic coil 110 is at the target site or location. For detachment, the puller 114 is advanced distally over the pusher 102 until it engages the detachment coupler 112. The puller 114 is held in place against the detachment coupler 112 while the pusher wire 106 is pulled proximally.

FIG. 6 is a sectional view showing the detachment tip 108 detached from the implant 104. When the pusher wire 106 is pulled proximally, the detachment tip 108 is withdrawn from the detachment coupler 112. The detachment method allows the user to mechanically control the disengagement of the implant 104 to the pusher 102. Upon implant detachment, the pusher and pull wire are retrieved. The pusher wire 106, detachment tip 108 and puller 114 are pulled proximally and removed from the patient.

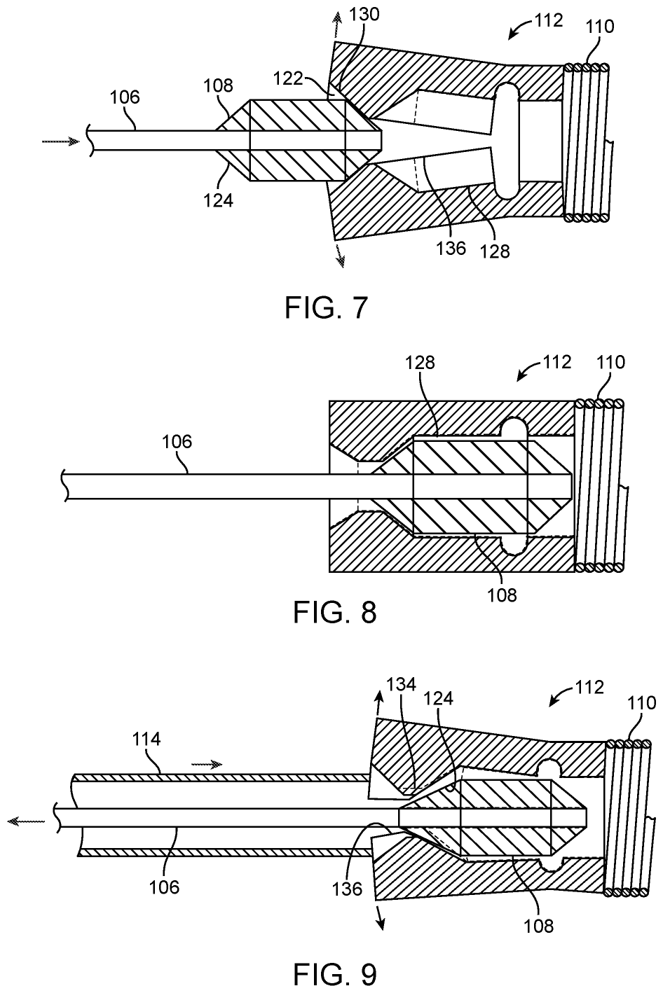

FIGS. 7-9 are sectional views showing the connect/disconnect features of the detachment coupler mechanism 100 that are configured to couple the detachment tip 108 of the pusher 102 with the detachment coupler 112 of the implant 104.

FIG. 7 shows the detachment tip 108 being coupled with the detachment coupler 112. During coupling, the detachment tip 108 is inserted into the opening 116 and the tapered distal end 122 engages the proximal ramped portion 130 of the detachment coupler 112 and the “T” cut 136 opens and expands the inlet 126 to allow the detachment tip 108 to enter the detachment tip cavity 128. Once the detachment tip 108 is within the detachment tip cavity 128, the inlet 126 returns to the closed position to retain the detachment tip 108 during implant delivery.

FIG. 8 shows the implant delivery configuration of the detachment coupler mechanism 100. During delivery of the implant 104, the detachment tip 108 may rotate freely within the detachment tip cavity 128 and does not require an external force to remain secured within the detachment tip cavity 128.

FIG. 9 shows detachment of the detachment tip 108 from the detachment tip cavity 128. The puller 114 is configured to slide distally over the pusher wire 106 and detachment tip 108 until a distal end engages the proximal side of the detachment coupler 112. The puller 114 is held against the detachment coupler 112 to keep it from moving while the pusher wire 106 and the detachment tip 108 are pulled proximally. During proximal movement of the detachment tip 108, the tapered proximal end 124 contacts the distal ramped portion 134 and the “T” cut 136 opens and expands the inlet 126 to allow the detachment tip 108 to exit the detachment tip cavity 128.

The implant assembly is configured to advance the implant 104 through a catheter to the target site or location with the pusher 102. At the target site or location, the implant 104 is detached from the pusher 102 at the detachment mechanism 100.

In some embodiments the pushers 102 and implants 104 may be selected from an assortment of pushers 102 and implants 104 having various properties for different procedures. The pushers 102 may include pusher wires 106 having different flexibility, from flexible for tortuous paths to stiff for direct paths, and detachment tips 106 having different sizes, shapes and/or made of different materials. The implants 104 may include embolic coils 110 of different sizes and/or shapes, and a detachment coupler configured to match the detachment tip 108.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A detachment coupler mechanism comprising:
an implant having an embolic coil coupled with a detachment coupler having one or more slots or cuts, the detachment coupler includes a detachment tip cavity with a proximal opening coupled to the one or more slots or cuts configured to allow the proximal opening to flex open and close;
a pusher having a pusher wire coupled with a distal detachment tip configured for insertion through the proximal opening and retainment within the detachment tip cavity, wherein the proximal opening is configured to flex open during insertion of the detachment tip and flex closed when the detachment tip body is within the detachment tip cavity; and
a puller configured to assist in detaching the detachment tip from the detachment coupler.

2. The detachment coupler mechanism of claim 1, wherein the one or more slots or cuts includes a “T” cut having a length L, width W and height H, wherein the force/friction strength of the flex for the proximal opening may be modified by increasing or decreasing the length L, width W and height H of the “T” cut.

3. The detachment coupler mechanism of claim 2, wherein a shorter “T” cut increases the force/friction strength and a longer “T” cut decreases the force/friction strength.

4. The detachment coupler mechanism of claim 1, wherein the puller is configured to apply a distal force against the implant coupler while the push wire applies a proximal force to withdraw the detachment tip from the detachment tip cavity.

5. The detachment coupler mechanism of claim 4, wherein the proximal opening is configured to flex open during withdrawal of the detachment tip and flex closed when the withdrawal of the detachment tip is complete.

6. The detachment coupler mechanism of claim 1, wherein the detachment includes a detachment tip body having a tapered distal end and a tapered proximal end.

7. The detachment coupler mechanism of claim 1, wherein the proximal opening includes a proximal ramped portion, a reduced diameter portion, and a distal ramped portion coupled to the detachment tip cavity.

8. The detachment coupler mechanism of claim 1, wherein inserting the detachment tip within the detachment tip cavity permits axial movement of the pusher and implant.

9. The detachment coupler mechanism of claim 1, wherein the detachment tip is configured to rotate freely within the detachment coupler and does not require an external force to remain secured.

10. A detachment coupler mechanism comprising:
a detachment coupler having a detachment tip cavity with a proximal opening coupled to one or more slots or cuts configured to allow the proximal opening to flex;
a pusher having a pusher wire with a distal detachment tip configured for insertion through the proximal opening and retainment within the detachment tip cavity, wherein one or more slots or cuts flex the proximal opening open during insertion of the detachment tip and flex closed when the detachment tip body is within the detachment tip cavity; and
a puller configured to apply a distal force against the detachment coupler to assist in detaching the detachment tip from the detachment tip cavity, wherein when the puller applies the distal to the detachment coupler, the push wire can apply an opposite proximal force to withdraw the detachment tip from the detachment tip cavity.

11. The detachment coupler mechanism of claim 10, wherein during withdrawal of the detachment tip the proximal opening is configured to flex open during withdrawal of the detachment tip the one or more slots or cuts flex open the proximal opening and flex closed when the withdrawal of the detachment tip is complete.

12. The detachment coupler mechanism of claim 10, wherein the one or more slots or cuts includes a “T” cut having a length L, width W and height H, wherein the force/friction strength of the flex for the proximal opening may be modified by increasing or decreasing the length L, width W and height H of the “T” cut.

13. The detachment coupler mechanism of claim 12, wherein a shorter “T” cut increases the force/friction strength and a longer “T” cut decreases the force/friction strength.

14. The detachment coupler mechanism of claim 10, wherein the detachment tip includes a detachment tip body having a tapered distal end and a tapered proximal end.

15. The detachment coupler mechanism of claim 10, wherein the proximal opening includes a proximal ramped portion, a reduced diameter portion, and a distal ramped portion coupled to the detachment tip cavity.

16. The detachment coupler mechanism of claim 10, wherein inserting the detachment tip within the detachment tip cavity permits axial movement of the pusher and implant.

17. The detachment coupler mechanism of claim 10, wherein the detachment tip is configured to rotate freely within the detachment coupler and does not require an external force to remain secured.

18. A detachment coupler mechanism comprising:
an implant having an embolic coil coupled with a detachment coupler having:
one or more “T” slots or cuts having a length L, width W and height H, wherein the force/friction strength of the flex for the proximal opening may be modified by increasing or decreasing the length L, width W and height H of the “T” slots or cuts;
a detachment tip cavity with a proximal opening coupled to the one or more “T” slots or cuts configured to allow the proximal opening to flex open and closed;
a pusher having a pusher wire coupled with a distal detachment tip configured for insertion through the proximal opening and retainment within the detachment tip cavity, wherein the distal detachment tip is configured to flex open the proximal opening during insertion of the detachment tip and flex closed when the detachment tip body is within the detachment tip cavity; and
a puller configured to apply a distal force against the detachment coupler to assist in detaching the detachment tip from the detachment tip cavity, wherein when the puller applies the distal to the detachment coupler, the push wire can apply an opposite proximal force to withdraw the detachment tip from the detachment tip cavity.

19. The detachment coupler mechanism of claim 18, wherein inserting the detachment tip within the detachment tip cavity permits axial movement of the pusher and implant and the detachment tip is configured to rotate freely within the detachment coupler and does not require an external force to remain secured.

20. The detachment coupler mechanism of claim 18, wherein the one or more slots or cuts includes a “T” cut having a length L, width W and height H, wherein the force/friction strength of the flex for the proximal opening may be modified by increasing or decreasing the length L, width W and height H of the “T” cut.
